(19) United States
(12) Patent Application Publication
(10) Pub. No.: US 2017/0027952 A1
(43) Pub. Date: Feb. 2, 2017

(54) ASSOCIATION BETWEEN 3-[(3--2-OXO-2,3-DIHYDRO-1H-INDOL-5-YL)METHYL]-1,3-THIAZOLIDINE-2,4-DIONE AND AN EGFR TYR KINASE INHIBITOR

(71) Applicant: LES LABORATOIRES SERVIER, SURESNES CEDEX (FR)

(72) Inventors: Michael BURBRIDGE, Verneuil Sur Seine (FR); Valérie CATTAN, Paris (FR); Anne JACQUET-BESCOND, Le Plessis Robinson (FR)

(21) Appl. No.: 15/219,691

(22) Filed: Jul. 26, 2016

(30) Foreign Application Priority Data
Jul. 31, 2015 (FR) 1557412

(51) Int. Cl. A61K 31/5377; A61K 31/517

(52) U.S. Cl. CPC A61K 31/5377; A61K 31/517

(57) ABSTRACT

Association between 3-[(3-{[4-(4-morpholinylmethyl)-1H-pyrrol-2-yl]methylene}-2-oxo-2,3-dihydro-1H-indol-5-yl)methyl]-1,3-thiazolidine-2,4-dione of formula (I):

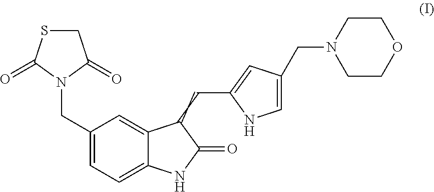 [SMILES: O=C1Nc2ccc(CN3C(=O)CSC3=O)cc2C1=Cc1cc(CN2CCOCC2)c[nH]1]

or a Z or E isomer thereof and/or an addition salt thereof with a pharmaceutically acceptable acid or base, and a human epidermal growth factor receptor (EGFR) tyrosine kinase inhibitor.

Medicinal products containing the same which are useful in treating non-small cell lung cancer.

The present invention relates to a new association between 3-[(3-{[4-(4-morpholinylmethyl)-1H-pyrrol-2-yl]methylene}-2-oxo-2,3-dihydro-1H-indol-5-yl)-methyl]-1,3-thiazolidine-2,4-dione of formula (I):

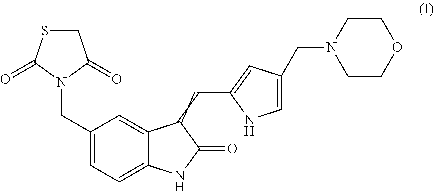 [SMILES: O=C1Nc2ccc(CN3C(=O)CSC3=O)cc2C1=Cc1cc(CN2CCOCC2)c[nH]1]

or a Z or E isomer thereof and/or an addition salt thereof with a pharmaceutically acceptable acid or base, and a human epidermal growth factor receptor (EGFR) tyrosine kinase inhibitor for the treatment of non-small cell lung cancer, more especially in patients who are resistant to an EGFR tyrosine kinase inhibitor.

Non-small cell lung cancer is today the leading cause of death from cancer in the world (Goldstraw, P., D. Ball, J. R. Jett, C. T. Le, E. Lim, A. G. Nicholson, and F. A. Shepherd, 2011, Non-small-cell lung cancer: Lancet, v. 378, no. 9804, p. 1727-1740; Jemal, A., F. Bray, M. M. Center, J. Ferlay, E. Ward, and D. Forman, 2011, Global cancer statistics: CA Cancer J Clin, v. 61, no. 2, p. 69-90). At the time of diagnosis, the majority of patients have an advanced pathology with a one-year survival rate of 30% and a 5-year survival rate of 10% (U.S. National Institutes of Health, National Cancer institute http://seer.cancer.gov/archive/csr/1975_2011/results_merged/topic_delaygraphs_overview. pdf; http://www.cancerresearchuk.org/cancer-info/cancerstats/types/lung/survival/lung-cancer-survival-statistics). Activating mutations of the EGFR gene lead to oncogene addiction, that is to say the cancer cell becomes dependent upon that anomaly for its growth and survival. Such mutations are frequent in adenocarcinoma of the lung with 15% of cases in Caucasian patients and 40-50% of cases in Asian patients (Shigematsu, H. et al., 2005, Clinical and biological features associated with epidermal growth factor receptor gene mutations in lung cancers: J Natl Cancer Inst, v. 97, no. 5, p. 339-346). In patients having a mutation of the EGFR gene, EGFR tyrosine kinase inhibitors significantly delay the progression of the disease as compared with chemotherapy and are considered to be the benchmark treatment. Current treatments on the market are inter alia gefitinib and erlotinib for first-generation inhibitors and afatinib for second-generation inhibitors, each generation targeting active mutations of EGFR. Unfortunately, most patients relapse after several months of treatment through the acquisition of other genetic and protein alterations which are capable of producing resistance to EGFR tyrosine kinase inhibitors. Several resistance mechanisms have been identified, and a new mutation of the EGFR receptor (T790M: substitution in position 790 of a threonine by a methionine) especially is found in the majority of patients who are resistant to treatment. Following resistance to EGFR tyrosine kinase inhibitors, the prognosis becomes very poor and patients are offered chemotherapy with a low efficacy rate. In this context, the search for new therapeutic alternatives in non-small cell lung cancer, and especially in patients who are resistant to EGFR tyrosine kinase inhibitors, with a view to improving progression-free survival, continues to be a current issue. In particular, resensitising patients who are resistant to EGFR tyrosine kinase inhibitors constitutes a strong therapeutic strategy for exploration. Third-generation EGFR tyrosine kinase inhibitors are currently being developed for acting specifically in patients who have acquired a secondary mutation such as T790M, for example, and appear to restore the activity of the treatment. Other alternatives which act on resistance pathways other than the T790M mutation and/or which involve other cell receptors are still necessary and very long-awaited by patients, and may especially be associated with third-generation inhibitors.

In the following and unless specified otherwise, “3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione” is understood as meaning “3-[(3-{[4-(4-morpholinyl-methyl)-1H-pyrrol-2-yl]-methylene}-2-oxo-2,3-dihydro-1H-indol-5-yl)methyl]-1,3-thiazolidine-2,4-dione, and its Z or E isomers and/or addition salts with a pharmaceutically acceptable acid or base”.

3-[(3-{[4-(4-Morpholinylmethyl)-1H-pyrrol-2-yl]methylene}-2-oxo-2,3-dihydro-1H-indol-5-yl)methyl]-1,3-thiazolidine-2,4-dione is a potent inhibitor of the migration of cancer cells which can be used especially for the treatment of cancers and especially of metastatic solid tumours. It is described in patent applications WO2011/015728 and WO2015/004395.

According to the invention, it has been shown that the effects of 3-{[(3-{[4-(4-morpholinylmethyl)-1H-pyrrol-2-yl]methylene}-2-oxo-2,3-dihydro-1H-indol-5-yl]methyl}-1,3-thiazolidine-2,4-dione allowed the resistance to EGFR tyrosine kinase inhibitors to be removed in animal models previously treated with such an inhibitor.

These effects allow the use of the association of 3-{[(3-{[4-(4-morpholinylmethyl)-1H-pyrrol-2-yl]methylene}-2-oxo-2,3-dihydro-1H-indol-5-yl]methyl}-1,3-thiazolidine-2,4-dione and an EGFR tyrosine kinase inhibitor in the treatment of non-small cell lung cancers to be envisaged, especially in patients for whom progression of the disease or a relapse has been observed in spite of the treatment.

More especially in the association according to the invention, the 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione is in the form of the Z isomer.

Preferably in the association according to the invention, the 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione is in the form of a salt, especially a hydrochloride or a mesylate.

Yet more advantageously, the association according to the invention comprises 3-[((3Z)-3-{[4-(4-morpholinyl-methyl)-1H-pyrrol-2-yl]methylene}-2-oxo-2,3-dihydro-1H-indol-5-yl)methyl]-1,3-thiazolidine-2,4-dione mesylate.

Among the EGFR tyrosine kinase inhibitors according to the invention there may be mentioned erlotinib, gefitinib and afatinib for the first- and second-generation inhibitors and AZD9291 (osimertinib) or rociletinib for the third-generation inhibitors.

According to an advantageous embodiment, the EGFR tyrosine kinase inhibitor of the association according to the invention is N-(3-ethynylphenyl)-6,7-di(2-methoxyethoxy)-quinazolin-4-amine or erlotinib of formula (II):

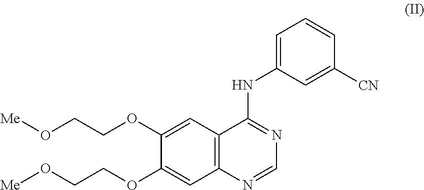 [SMILES: COCCOc1cc2ncnc(Nc3cccc(C#N)c3)c2cc1OCCOC]

or an addition salt thereof with a pharmaceutically acceptable acid or base, and especially its hydrochloride.

According to another advantageous embodiment, the EGFR tyrosine kinase inhibitor of the association according to the invention is N-(3-chloro-4-fluoro-phenyl)-7-methoxy-6-(3-morpholin-4-ylpropoxy)quinazolin-4-amine or gefitinib of formula (III):

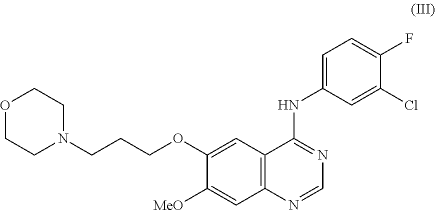 [SMILES: COc1cc2ncnc(Nc3ccc(F)c(Cl)c3)c2cc1OCCCN1CCOCC1]

or an addition salt thereof with a pharmaceutically acceptable acid or base.

Preferably, the association according to the invention comprises the Z isomer of 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione or a pharmaceutically acceptable salt thereof, with gefitinib or a pharmaceutically acceptable salt thereof.

Yet more especially, the invention relates to the association between 3-[((3Z)-3-{[4-(4-morpholinyl-methyl)-1H-pyrrol-2-yl]methylene}-2-oxo-2,3-dihydro-1H-indol-5-yl)-methyl]-1,3-thiazolidine-2,4-dione mesylate and gefitinib or a pharmaceutically acceptable salt thereof.

The invention relates also to pharmaceutical compositions comprising the association between 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione and an EGFR tyrosine kinase inhibitor in combination with one or more pharmaceutically acceptable excipients.

The invention relates also to the use of said pharmaceutical compositions in the treatment of non-small cell lung cancer, more especially in patients who are resistant to an EGFR tyrosine kinase inhibitor.

Among the pharmaceutical compositions according to the invention there may be mentioned more especially those that are suitable for administration by the oral, parenteral, intramuscular and intravenous, per- or trans-cutaneous, nasal, rectal, perlingual, ocular or respiratory route and more specifically tablets, drages, sublingual tablets, gelatin capsules, glossettes, capsules, lozenges, injectable preparations, aerosols, eye or nasal drops, suppositories, creams, ointments, dermal gels, etc.

In a preferred embodiment, the 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione is administered in oral form.

In addition to the 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione and the EGFR tyrosine kinase inhibitor, the pharmaceutical compositions according to the invention comprise one or more excipients or carriers chosen from diluents, lubricants, binders, disintegrators, stabilisers, preservatives, absorbents, colourings, sweeteners, flavourings, etc.

Examples which may be mentioned, without implying any limitation, include:
for the diluents: lactose, dextrose, sucrose, mannitol, sorbitol, cellulose, glycerin;
for the lubricants: silica, talc, stearic acid and its magnesium and calcium salts, polyethylene glycol;
for the binders: aluminium and magnesium silicate, starch, gelatin, tragacanth, methylcellulose, sodium carboxymethylcellulose and polyvinylpyrrolidone;
for the disintegrators: agar, alginic acid and its sodium salt, effervescent mixtures.

The compounds of the association can be administered simultaneously or in succession. The corresponding pharmaceutical compositions can permit the immediate or delayed release of the active ingredients. Moreover, the compounds of the association can be administered in the form of two separate pharmaceutical compositions, each comprising one of the active ingredients, or alternatively in the form of a single pharmaceutical composition in which the active ingredients are mixed.

The dosage used varies according to the sex, age and weight of the patient, the administration route, the nature of the cancer and of any associated treatments and ranges from 300 to 1500 mg of equivalents of free base of 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione per day, and more preferably from 400 to 800 mg of equivalents of free base per day, and yet more especially from 500 to 600 mg of equivalents of free base per day. The dose of the EGFR tyrosine kinase inhibitor will be equal to that used when it is administered on its own or less. By way of example, in the case of gefitinib, the dose administered is 250 mg per day. For erlotinib, it is from 25 to 150 mg per day.

Pharmaceutical Composition

| | | |
|---|---|---|
| 1000 tablets containing 100 mg of equivalents of free base of | 121 | g |
| 3-[3-{[4-(4-morpholinylmethyl)-1H-pyrrol-2- | | |
| yl]methylene}-2-oxo-2,3-dihydro-1H-indol-5-yl)methyl]- | | |
| 1,3-thiazolidine-2,4-dione methanesulphonate, Z isomer | | |
| Sodium starch glycolate | 20 | g |
| Maize starch | 133 | g |
| Lactose monohydrate | 357 | g |
| Magnesium stearate | 6.7 | g |
| Silica | 1.3 | g |
| Povidone | 46.6 | g |

Preclinical Studies

A) Cell Viability Test on the Erlotinib-Resistant Cell Line HCC827

A cell viability test allowing the anti-proliferative ability of the anti-tumour compounds to be measured was used. The chosen cell line is line HCC827, a non-small cell lung cancer line that depends upon EGFR for its survival. The parameter used is the IC50, that is to say the concentration of product that inhibits 50% of the cell proliferation in comparison with untreated control cells. The cells are seeded (150 μl) at the appropriate density in the wells of 96-well plates 2 days prior to the experiment. One column contains the untreated control cells representing 100% proliferation. The others are incubated with the test products for 4 doubling times. The median inhibitory concentration of the EGFR tyrosine kinase inhibitor erlotinib for the cell viability of the line HCC827 is 10 nM. Acquired resistance to erlotinib is generated by chronic exposure of the line HCC827 to erlotinib: the cells are exposed to erlotinib at a dose of 1 μM in the culture medium until the doubling time stabilises, that is to say approximately 2 months. The median inhibitory concentration of erlotinib for the cell viability of the resistant line HCC827 is then approximately 1000 times higher at 11.5 μM. The resistant cells are then exposed to 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione hydrochloride at a concentration of 100 nM in combination with increasing doses of erlotinib. 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione hydrochloride on its own has no effect on the viability. In combination, the median inhibitory concentration of erlotinib on the resistant line HCC827 reverts to approximately that of the non-resistant line HCC827 at 3.8 nM.

This result shows that 3-[(3-{[4-(4-morpholinyl-methyl)-1H -pyrrol-2-yl]methylene}-2-oxo-2,3-dihydro-1H -indol-5-yl)methyl]-1,3-thiazolidine-2,4-dione is capable of restoring sensitivity to an EGFR tyrosine kinase inhibitor in a non-small lung cancer cell line that is resistant to such an inhibitor.

B) inhibition of the Growth of the Erlotinib-Resistant HCC827 Tumour

Line HCC827, a non-small cell lung line, rendered resistant to erlotinib in vitro, was grafted at a subcutaneous location onto female SCID mice in an amount of 5·10⁶ cells per mouse. The tumours were allocated at random into groups of eight mice when the tumour volume had reached approximately 200 mm³. Daily treatments with compound A (3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione hydrochloride) at a dose of 50 mg/kg and with erlotinib at a dose of 12.5 mg/kg were administered orally (carriers=ammonium acetate/HEC and PEG300/ethanol/water buffers, respectively) over a period of 19 days, as indicated by the triangles on FIG. 1 below. The tumour volumes were measured two to three times each week using a sliding caliper. The median tumour volumes with the interquartile ranges are recorded on the graph.

At the end of the treatments, on d19, inhibition of the growth after treatment with the compounds on their own is 65% for erlotinib and 83% for 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione hydrochloride (compound A). When the two compounds are used in combination, the tumours regress completely, and this regression persists over time, once treatment has been stopped, until d30. The synergy observed between the two products is statistically significant over the study period (p<0.001).

Clinical Study

Patients with a non-small cell lung cancer develop resistance to treatment with an EGFR tyrosine kinase inhibitor (gefitinib, erlotinib, afatinib, osimertinib or rociletinib) and are no longer sensitive to the treatment, and the disease progresses.

A proof of concept study is in progress in order to confirm the results observed in the preclinical studies and show that 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione is capable of restoring sensitivity to an EGFR inhibitor, in the present case gefitinib, in patients suffering from a non-small cell lung cancer who have become resistant. The patients are included according to their molecular profile. This study includes a phase I with the objective of evaluating the tolerance profile and determining the recommended dose for the continued development. Approximately 20 patients will be included. During this phase, the patients will be treated in 28-day cycles with a dose of 400, 500 or 600 mg per day of equivalents of free base of 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione in combination with 250 mg per day of gefitinib. The treatment will be maintained until the disease progresses. At the end of this phase, a phase II will be initiated, with the objective of evaluating the activity of the combination between 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione and gefitinib. This phase II will include approximately 150 patients. The tumours will be evaluated every 2 months. Patients will be treated in 28-day cycles with the recommended dose of 3-[(3-{[4-(4-morpholinyl-methyl)-1H-pyrrol-2-yl]methylene}-2-oxo-2,3-dihydro-1H-indol-5-yl)methyl]-1,3-thiazolidine-2,4-dione defined in phase I in combination with 250 mg of gefitinib per day.

### BRIEF DESCRIPTION OF THE DRAWINGS

FIG. 1 shows the results of an in vitro study to evaluate inhibition of the growth of the erlotinib-resistant HCC827 tumour.

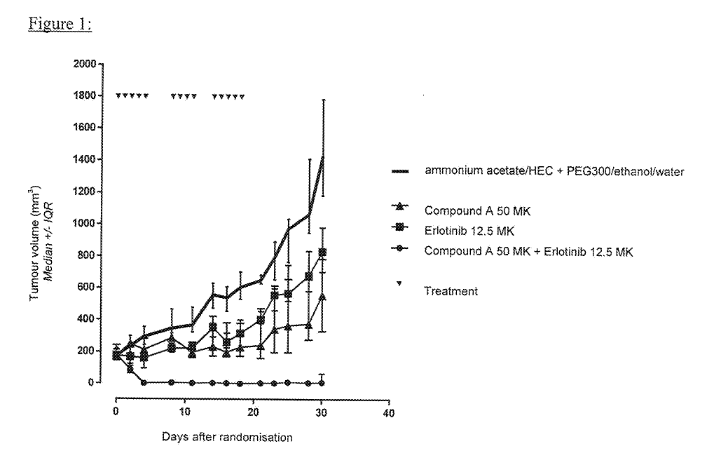

1. A combination between 3-[(3-{[4-(4-morpholinylmethyl)-1H-pyrrol-2-yl]methylene}-2-oxo-2,3-dihydro-1H-indol-5-yl)methyl]-1,3-thiazolidine-2,4-dione of formula (I): 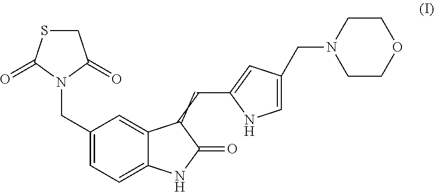 [SMILES: O=C1Nc2ccc(CN3C(=O)CSC3=O)cc2C1=Cc1cc(CN2CCOCC2)c[nH]1]
or a Z or E isomer thereof and/or an addition salt thereof with a pharmaceutically acceptable acid or base, and a human epidermal growth factor receptor (EGFR) tyrosine kinase inhibitor.

2. The combination according to claim 1, wherein the 3-[(3-{[4-(4-morpholinylmethyl)-1H-pyrrol-2-yl]methylene}-2-oxo-2,3-dihydro-1H-indol-5-yl)methyl]-1,3-thiazolidine-2,4-dione is in the form of the Z isomer.

3. The combination according to claim 1, wherein the 3-[(3-{[4-(4-morpholinylmethyl)-1H-pyrrol-2-yl]methylene}-2-oxo-2,3-dihydro-1H-indol-5-yl)methyl]-1,3-thiazolidine-2,4-dione is in the form of a hydrochloride.

4. The combination according to claim 1, wherein 3-[(3-{[4-(4-morpholinylmethyl)-1H-pyrrol-2-yl]methylene}-2-oxo-2,3-dihydro-1H-indol-5-yl)methyl]-1,3-thiazolidine-2,4-dione is in the form of a mesylate.

5. The combination according to claim 1, wherein the EGFR tyrosine kinase inhibitor is gefitinib or erlotinib.

6. A method of treating non-small cell lung cancer in a subject in need thereof, comprising administration of an effective amount of the combination according to claim 1.

7. A method of treating non-small cell lung cancer in patients who are resistant to an EGFR tyrosine kinase inhibitor, comprising administration of an effective amount of the combination according to claim 1.

8. A pharmaceutical composition comprising as active ingredient the combination according to claim 1, in combination with one or more pharmaceutically acceptable excipients.

9. A method of treating non-small cell lung cancer in a subject in need thereof, comprising administration of an effective amount of the composition according to claim 8.

10. A method of treating non-small cell lung cancer in patients who are resistant to an EGFR tyrosine kinase inhibitor, comprising administration of an effective amount of the composition according to claim 8.
